(19) United States
(12) Patent Application Publication
(10) Pub. No.: US 2016/0106893 A1
(43) Pub. Date: Apr. 21, 2016

(54) Surgical aspirator probe with adaptive tip

(71) Applicant: Jaime Zacharias, Santiago (CL)

(72) Inventor: Jaime Zacharias, Santiago (CL)

(21) Appl. No.: 14/519,561

(22) Filed: Oct. 21, 2014

(51) Int. Cl. A61M 1/00; A61F 9/007

(52) U.S. Cl. CPC A61M 1/0088; A61F 9/00736; A61F 2250/0006; A61F 2250/0008

(57) ABSTRACT

A surgical tip having a soft adaptive distal end that conforms to the irregular cross sectional contours of lens fragments, this construction allowing better occlusion of the tip distal end by adapting to the irregular shapes of the lens fragments thus improving the efficiency of the lensectomy probe by enhancing vacuum build up, reducing the total irrigant volume required to complete the lensectomy process and protects the lens capsule from accidental rupture said soft tip being usable with vibratory based, laser-based and water-jet based lensectomy handpieces as well as lens aspiration cannula.

### CROSS-REFERENCE TO RELATED APPLICATION

This application claims the benefit of U.S. Provisional Application No. 61/893854 filed Oct. 21, 2013.

### BACKGROUND OF THE INVENTION

This invention relates generally to the field of cataract surgery and more particularly to a handpiece tip for practicing the irrigation/aspiration technique of cataract fragments removal.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL). Also the crystalline lens can be exchanged prior to development of significant cataract for the correction of refractive defects such as hyperopia, astigmatism and myopia by replacement of an intraocular lens (IOL).

In the United States, the majority of lensectomy procedures are performed by a surgical technique called phacoemulsification. During this procedure, a thin phacoemulsification cutting tip is inserted into the target lens and vibrated ultrasonically. The vibrating tip liquifies or emulsifies the lens so that the lens may be aspirated out of the eye. The lens material, once removed, is replaced by an artificial lens typically placed inside the crystalline lens capsular bag.

New advances in ultrafast (UF) laser power delivery into ocular tissues can allow three dimensional “cutting” of the crystalline lens material into small segments, as part of a procedure known as Femto Laser Assisted Cataract Surgery (FLACS) infrared femtosecond lasers.

A 3D laser scanner delivers UF laser pulses within the lens material to produce physical disruption of the lens substance along the path of the high frequency UF laser pulses. UF laser trajectory can be programmed to sum up to produce cuts in tissue of accurate dimensions and location within the lens volume, usually guided by an imaging system such as OCT. Cuts can be superimposed to accurately segment the lens material into a plurality small fragments. As a mode of example, cubic-like fragments, typically between 0.2×0.2×0.2 mm to 0.5×0.5×0.5 mm, can be obtained, for further ultrasonic emulsification and removal by aspiration from within the eye by a lensectomy probe.

In use, the ends of this lensectomy tip and the optional irrigating sleeve are inserted into a small incision of predetermined width in the cornea, sclera, or other location. The lensectomy tip is vibrated along one or more of its axis using sonic or ultrasonic power, thereby emulsifying the selected tissue in situ.

The hollow bore of the lensectomy tip communicates with the bore in the horn that in turn communicates with the aspiration line from the handpiece to the console. A reduced pressure or vacuum source in the console draws and holds the lens fragments and then aspirates the emulsified tissue from the eye through the open end of the lensectomy tip, the lensectomy probe and horn bores and the aspiration line and into a collection device.

The aspiration of emulsified tissue is aided by a saline flushing solution or irrigant that can be injected into the surgical site through the small annular gap between the inside surface of an irrigating sleeve and the lensectomy tip or using a secondary irrigating instrument using a secondary incision.

It has been noted that with the introduction of FLACS to fragment the lens material into small pieces, the requirement of ultrasonic probe vibration have been significantly reduced, to an extent where the fragments from many soft to medium hardness lenses can be totally removed by aspiration only, without any use of ultrasonic tip vibration. This reduction or elimination of the use of ultrasound has been correlated with improved visual outcomes and reduced complications.

Aspiration of the FLACS generated lens fragments, as well as other residual lens material, is typically done through the metallic thus ultrasonic probe, with a rigid un-deformable distal end which can have cutting edges for improved phacoemulsification efficiency. Some new designs have incorporated relatively rigid plastic polymers to shield the metal probe distal end to prevent damage to lens capsule and other ocular tissues by unseen burrs and spurs emerging from the metal tip. The contour of the distal end of ultrasonic lensectomy probes is typically rounded or elliptical.

Alternatively, the softer lens pieces can frequently be aspirated using an aspiration lensectomy probe or I/A probe. These aspiration probes have round shaped aspiration ports and are typically non-sharp, or blunted, sometimes highly polished for enhanced smoothness or with a sanded texture, again to limit the risk of lens capsule rupture. Some metallic aspiration probes can incorporate a distal end elastomer sleeve where the rounded aspiration port is located to avoid direct contact between the lens capsule and the probe metal.

Prior art lensectomy probes have aspiration distal ends of metallic or relatively stiff polymer composition with rounded aspiration ports of stable opening dimensions. Some probes can be covered by elastomeric sleeves with have rounded distal openings of relatively stable dimensions.

The rounded nature and the dimensional stability of the aspiration port opening of existing lensectomy probes can produce sub-optimal occlusions reducing the efficiency of the fragment removal process i.e. leading to increased procedure time, excessive fluid consumption, increased turbulence and an unnecessary need for ultrasound to promote better occlusions, all of these eventually leading to a reduction in the quality of the surgical outcomes. This is particularly relevant when facing the highly faceted small lens fragments produced by the new FLACS technology, such as for instance, when facing cubic-like lens fragments. The fact that the lens fragment facets have poor three-dimensional matching with the stable sized, round edges of the aspiration port of the existing probes can have a negative impact.

Therefore, a need continues to exist for a lensectomy probe with a distal end having an adaptive aspiration port that can conform during aspiration to the irregular shapes and facets of the lens fragments being aspirated, in a way that occlusion is achieved faster, is tighter, and can be maintained during lens fragment aspiration by continuous adaptation of the aspiration port shape and dimensions with those of the lens fragment is being aspirated.

### BRIEF SUMMARY OF THE INVENTION

The present invention improves upon the prior art by providing a surgical

lensectomy probe with an aspiration port that can dynamically adapt to produce an improved fluidic seal around the irregular and evolving shapes presented by lens fragments while being aspirated. The energy required for the dynamic deformation and adaptation process of the aspirating tip is derived from the vacuum inside the aspiration line that induces a force between the lens fragments and the adaptive tip rim promoting sealing and more efficient action of vacuum to remove fragments.

In this way, when vacuum is applied, the aspiration port of the lensectomy probe of the present invention promotes the formation of a fluidic seal around the lens segments leading to faster and better occlusion by lens material, faster vacuum build up, reduced irrigant circulation and improved efficiency of a lens disrupting energy if required. The adaptive nature of the aspiration port of the lensectomy probe of the present invention leads to rapid occlusion which in turn improves lens segment grasping force. This force is also better sustained while a lens fragment evolves from an initial occlusion position until total aspiration.

Accordingly, one objective of the present invention is to provide a lensectomy probe that promotes rapid occlusion of the aspiration port by lens fragments including irregularly shaped and multifaceted lens fragments by promoting a fluidic seal around the fragment perimeter in contact with the aspiration port.

Another objective of the present invention is to provide a lensectomy probe that maintains the quality of the occlusion while aspirating these irregularly shaped and faceted lens fragments.

Another objective of the present invention is to provide a lensectomy probe with improved efficiency to maintain a grasping force at the aspiration port by dynamically adapting to varying fragment shape, position and orientation.

Another objective of the present invention is to provide a lensectomy probe that improves vacuum build up by rapid and effective occlusion of the adaptive aspiration port.

Another objective of the present invention is to provide a lensectomy probe that reduces irrigant circulation inside the eye chambers by improved occlusion by lens fragments.

Another objective of the present invention is to provide a lensectomy probe with improved lens fragment removing power provided by fast vacuum build-up by rapid and effective occlusion at the aspiration port

Another objective of the present invention is to provide a lensectomy probe with an improved lens capsule safety profile by preventing metallic and rigid polymers to enter in contact with the lens capsule.

These and other advantages and objectives of the present invention will become apparent from the detailed description and claims that follow.

### BRIEF DESCRIPTION OF THE DRAWINGS

FIG. 1 is a partial longitudinal cross-sectional view of a prior art aspirating handpiece tip with a lens fragment aspirated at the tip entrance.

FIG. 2 is a front view of the distal opening of a prior art aspirating handpiece tip with a lens fragment aspirated at the tip entrance.

FIG. 3 is a partial longitudinal cross-sectional view of the aspirating handpiece tip of the present invention with lens fragment aspirated at the tip entrance.

FIG. 4 is a front view of the distal opening of an aspirating handpiece tip of the present invention similar to FIG. 2, but illustrating the adaptation of the tip rim to a tri-facet shaped lens fragment during use.

FIG. 5 is a front view of the distal opening of an aspirating handpiece tip of the present invention similar to FIG. 4, but illustrating the adaptation of the tip rim to a tri-facet shaped lens fragment that has rotated during use.

FIG. 6 is a front view of the distal opening of an aspirating handpiece tip of the present invention similar to FIG. 2, but illustrating the adaptation of the tip rim to a tetra-facet shaped lens fragment during use.

FIG. 7 is a partial longitudinal cross-sectional view of the aspirating handpiece tip of the present invention with a lens fragment aspirated at the tip entrance showing deformation of the adaptive tip distal end deforming to seal the gaps with the irregularly shaped lens fragment.

FIG. 8 is another longitudinal partial cross-sectional view of the fragment being aspirated in FIG. 7 with the fragment being effectively deformed and disassembled while advancing inside the aspirating probe by the high vacuum achievable by the enhanced fluidic seal provided by the adaptive aspiration tip.

FIG. 9A Depicts a longitudinal partial cross section of another embodiment of the present invention where the adaptive probe tip is composed by a highly deformable elastomer membrane that will adapt and create a fluidic seal around lens fragments being aspirated here shown in the resting state.

FIG. 9B is a top side view of the embodiment shown in FIG. 9A

FIGS. 9C and 9D are respective views of the embodiment shown in FIGS. 9A and 9B being partially deformed by flow and prepared to conform an effective seal with lens particles aspirated into the port and attempting to pass through.

FIGS. 10A and 10B shows an alternative embodiment of the present invention where a membrane progressively thinning towards the distal rim constitutes an adaptive probe tip for improved fluidic seal and aspiration efficiency eventually detachable from a rigid aspirating probe.

FIG. 11 is a single piece embodiment similar to FIG. 10 where aspiration probe and adaptive tip are inseparable.

### DETAILED DESCRIPTION OF THE INVENTION

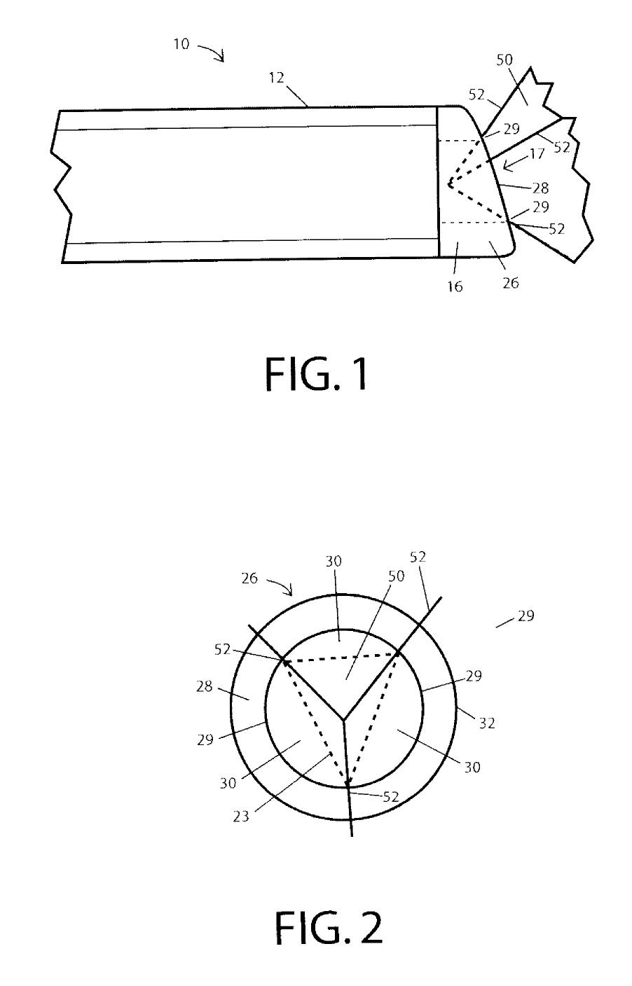

As best seen in FIG. 1 and FIG. 2, prior art lensectomy probe 10 generally consists in tube 12 made of metal or a rigid polymer. Distal tip 16 of tube 12 can be flared or belled. Distal tip 16 can include a rigid or elastomeric termination 26 conforming a stable aspiration port opening with a smooth rounded aspiration port rim 28 having an inner wall 29 shown in FIG. 2. As seen in views depicted in FIG. 1 and FIG. 2, when an irregularly shaped lens fragment 50 is drawn by vacuum inside tube 12 into the aspiration port 16 and termination 26, fluid aspiration across central aspiration channel 15 produces a fluid current through gaps 30 around fragment 50 walls 23 and distal port rim 28 inner edge 29. Fragment section corners 52 are stopped from advancing into tube 15 by inner edge 29 also promoting persistence of gaps 30. The fixed form nature of this prior art distal tip 16 impedes or delays occlusion of fragment-tip gaps 30 also retarding vacuum build-up leading to reduced efficiency and excessive fluid circulation.

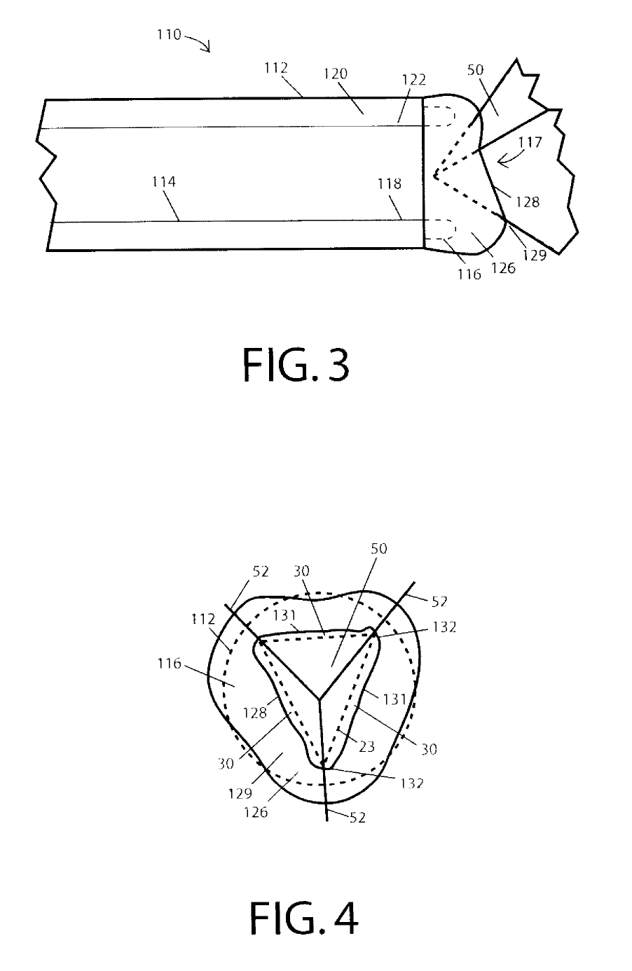

As best seen in FIG. 3, a lensectomy probe tip 110 of the present invention generally includes a tube 112 that can be straight or curved. Aspiration port 116 includes an elastomer portion 126 conforming a soft and deformable aspiration port rim 129 capable of forming an inner edge 128. As seen in the lateral view depicted in FIG. 3 and in the cross-section view depicted in FIG. 4, when an irregularly shaped lens fragment 50 with corners 52 is drawn by a fluid current or by a surgical maneuver into close contact with aspiration port 116 a fluid aspiration and a vacuum inside central aspiration channel 115 produces a pulling effect of the fragment toward deformable elastomer portion 126 with a force producing a contour deformation and adaptation of inner wall 128 of adaptive tip rim 129 and a 3D matching between the fragment perimeter shape and elastomeric rim 126. Fluid leaking gaps 30 between section 23 of fragment facets 50 and other irregularities are minimized by the seal provided by rim 129 inner edge 128 building a vacuum.

The deformable nature of elastomer portion 126 of aspiration port 116 changes its perimeter shape to adapt under the force produced by vacuum supplied across a fluidic path to aspiration channel 15 by a controllable vacuum source usually provided by a surgical console. This adaptation by deformation complements the diverse shapes of lens fragment 50 both circularly narrowing and expanding contributing to an improved seal of the fragment-tip gaps 30. This adaptive condition promoting tight fluidic seals between the probe rim and lens fragments of varying shape has a positive feedback effect. The tighter the seal, the higher the built vacuum, further enhancing the seal quality until the fragment brakes down and is aspirated through channel 15. This enhanced fluidic seal speeds up the process of vacuum build-up and increases efficiency reducing fluid circulation as fragments 50 are aspirated. The deformation process of elastomer portion 126 that contributes to adapt to the lens fragments variable and evolving section contour involves controlled inward, outward, advance and recession, bending and deformation according to the variable three-dimensional shape of the lens fragments 50 including facets 23, corners 52 and recesses.

Port 116 can have a diameter ranging between 0.1 and 2.0 mm. The amount of deformation departing from the resting state for elastomer portion 126 is typically in the range of 0.4 mm or less. According to fragment shapes, the inner edge 128 can have portions that displace inwards shown with 131 and other portions that displace outwards shown with 132 as well as limited forward and backward displacements.

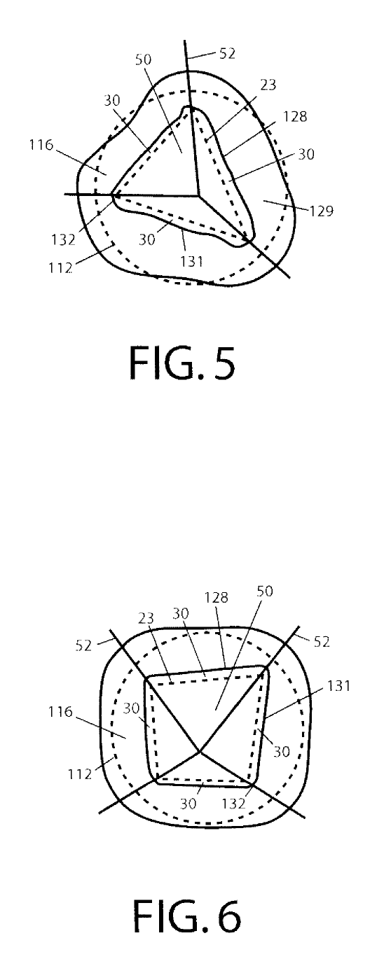

FIG. 5 illustrates the elastomer portion 126 of adaptive aspiration port 116 with port rim 129 inner wall 128 adapting to a CCW rotating lens fragment as compared to FIG. 4. It is shown that the tip rim 129 inner wall 128 deforms to adapt to the rotated lens fragment in a way that the inwardly bent portions 131 of inner wall 128 shown in FIG. 4 dynamically change to outwardly bent portions 132 and vice versa.

In FIG. 6 a four facet section lens fragment that could be a corner of a cubic shaped lens fragment produced by an UF laser treatment. Shown is as a mode of example with adaptive distal tip 116 inner wall 132 sealing the gaps 130 to promote occlusion, reduce flow, enhance vacuum build up and increase efficiency to remove lens fragments.

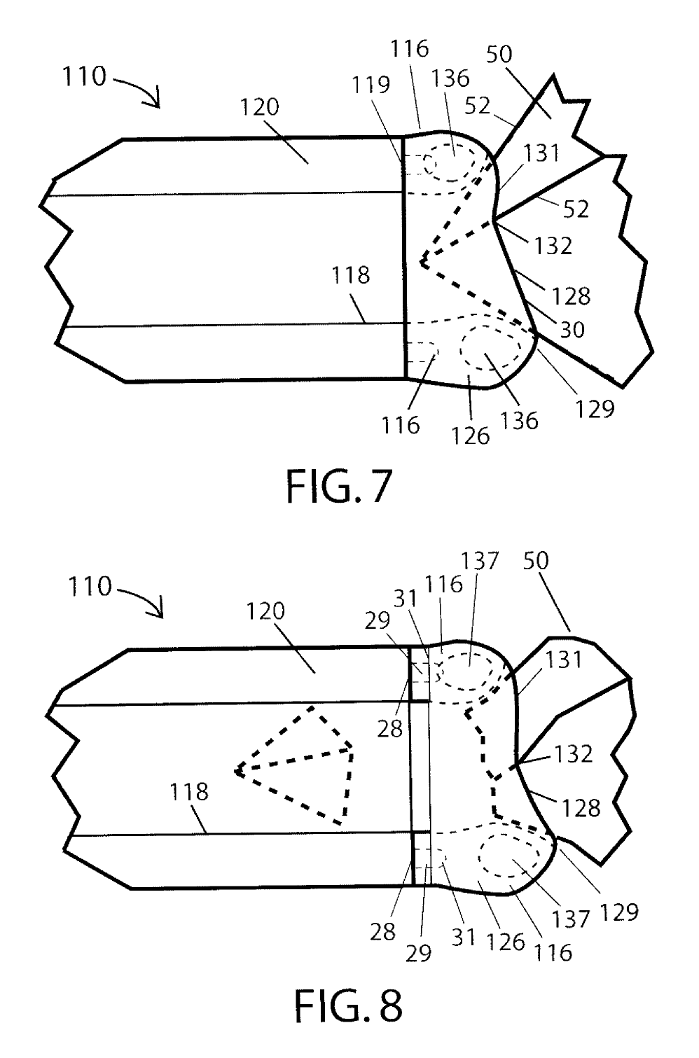

As depicted in FIG. 7 adaptive aspiration port 116 has elastomer portion 126 with a tip rim 129 inner wall 128 displaced inwards (portion 131) or outwards (portion 132) typically in the range of 0.4 mm. to adapt to the diverse possible shapes of lens fragments 50 and to effectively seal gaps 30. A differential polymerization process or a layered combination of elastomers producing different durometer readings can be incorporated with advantage to provide a range of suitable durometer readings such as shown portion 136 diametrically or axially across the elastomer portion 126 to improve adaptation to irregular lens fragment 150 shapes with improved gap 30 sealing properties. In this example, an annular embedded portion 136 of tip 126 is made of an elastomer with a lower durometer reading to provide enhanced deformability and adaptability to better adjust to changing shapes lens fragments than a homogeneous elastomer tip would do.

FIG. 8 depicts an alternative embodiment where adaptive aspiration port 116 has a more rigid elastomer portion 29 transforming into a softer elastomer portion 126 having rim 129 and inner wall 128 displacing inwardly or outwardly, forward or backward typically in the range of 0.4 mm to adapt to the diverse possible shapes of a lens fragment 50 and to effectively seal fluid leaking gaps 30. Also depicted in FIG. 8, one or more pockets 137 can be incorporated within elastomer portion 126 to further improve the adaptive properties of distal tip 116 to irregular lens fragment 50 section shapes. Pockets 137 can be filled with a gas, a biocompatible liquid or with a different durometer reading cured elastomer material than the one composing the body of elastomer portion 126.

The improved lens fragments 50 occlusion characteristics of the lensectomy probe 110 of the present invention promotes rapid and stable dynamic occlusions of aspiration port opening 117 acting in a cushion-like form adapting to irregularly shaped lens fragments 150. Also, the adaptive lensectomy probe 110 of the present invention improves the quality of the occlusion obtained when aspirating irregularly shaped lens fragments 50. The improved occlusion is particularly efficient to aspirate UF laser produced lens fragments, such as small cubes, that can have flat walls and corners. The improved vacuum build up is obtained by the rapid and effective occlusion provided by the adaptive aspiration port 116. Irrigant circulation is limited by the rapid and effective occlusion produced by the adaptive nature of distal tip 116.

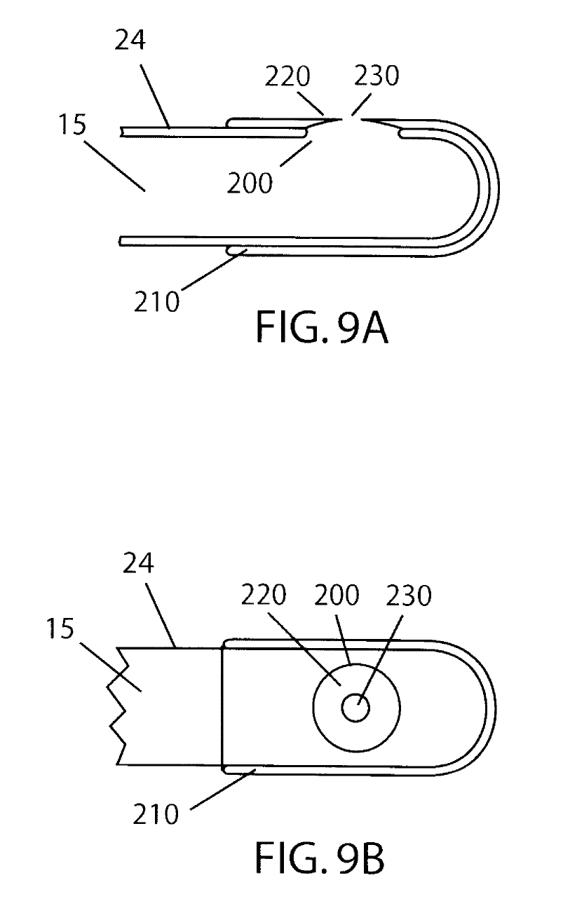

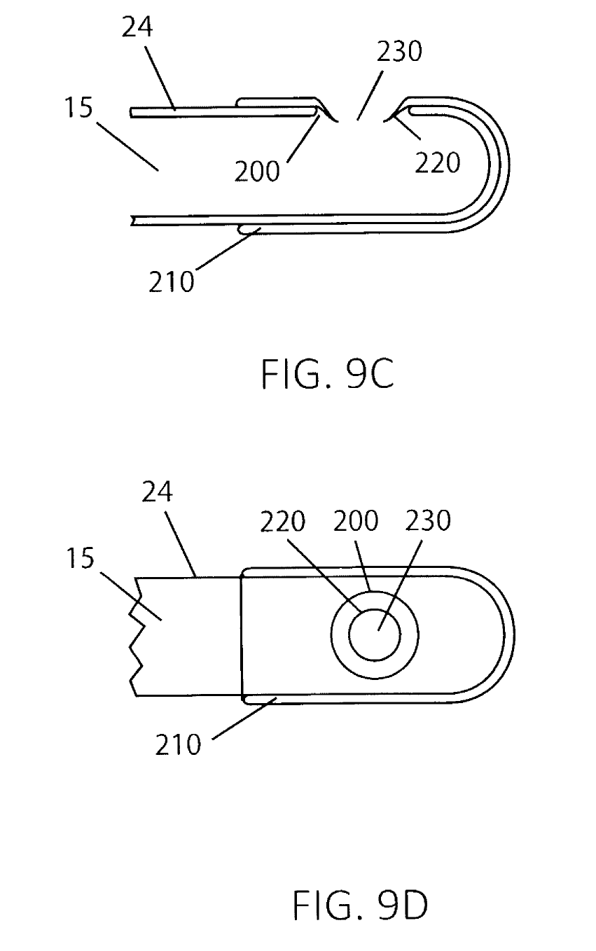

Shown in FIGS. 9A and 9B is an alternative embodiment shown in resting condition where an inner tube 12 has an opening 200. An elastomer portion 210 is adhered to tube 12 by suitable means, compression force, adhesive or other. Elastomer portion 210 has a section comprised by a deformable elastic membrane 220 located on top of opening 200. Membrane 220 with a circular perforation 230 centered with respect to underlying tube opening 200. When a vacuum is applied inside a tube 12 aspiration channel 15, a flow is created across perforation 230. FIGS. 9C and 9D show the embodiment from FIGS. 9A and 9B with a vacuum applied in aspiration channel 15 inside tube 12. As can be seen in a lateral cross section in FIG. 9C and in a lateral top view in FIG. 9D, the inflow produced by vacuum across perforation 230 produces an inward elongation of membrane 220 and a subsequent increase in diameter of perforation 230. In the same manner explained in FIGS. 3 to 8, irregularly shaped lens fragments drawn into contact with membrane 220 and perforation 230 will be attached and with increasing seal and adaptation, vacuum build up will forcefully aspirate the lens segments with high efficiency and minimal fluid leakage.

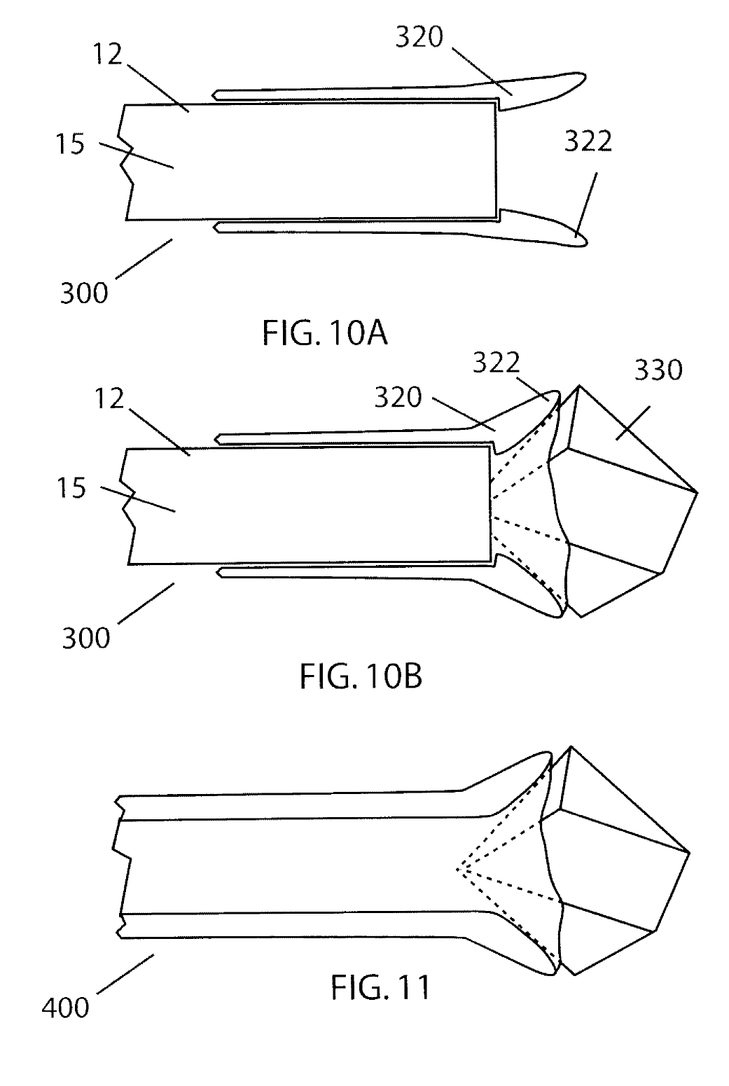

FIGS. 10A and 10B depict another embodiment of the present invention. In this embodiment the elastomeric tip 300 fit to an aspirating tube 12 with an inner channel 15 has a particular shape progressively narrowing and thinning toward the distal end. Typical dimensions are inner diameter 0.8 mm, outer diameter 1.1 mm, wall thickness at the base 320 of 300 microns, distal projection from base 1.2 mm and wall thickness at the distal end 200 of 50 microns. As seen in FIG. 10, adaptive tip 300 is in resting position. FIG. 10B depicts an example of the deformation and adaptation when a lens fragment 330 is being aspirated. Tip 300 encircles the perimeter of the lens fragment effectively sealing the periphery for enhanced vacuum build up and efficiency. FIG. 11 illustrates a single piece aspirating probe similar to FIG. 10.

The elastomer termination of the aspiration ports of the present invention provides the added benefit of a lensectomy probe with improved lens capsule safety characteristics.

This description is given for purposes of illustration and explanation. It will be apparent to those skilled in the relevant art that changes and modifications may be made to the invention described above without departing from its scope or spirit. For example, it will be recognized by those skilled in the art that the present invention may also be combined with ultrasonic, laser or rotatory powered lensectomy tips to enhance occlusion and vacuum build up to increase efficiency and to reduce fluid consumption improving the outcomes of the surgical procedures.

1. A lensectomy probe comprising: a) a controllable vacuum source; b) a probe distal end conforming an aspiration opening, said aspiration opening in fluid communication with aspiration means; c) the probe distal end with a rim including an elastic deformable portion circularly disposed around said aspiration opening in a way that said elastic deformable portion is adaptive to conform by pressure or vacuum to the irregular cross sections of lens tissue fragments providing faster and better occlusion of said tip aspiration opening by said lens tissue fragments.

2. The elastic deformable portion of claim 1 being composed of an elastomeric material such as silicone rubber.

3. The elastic deformable portion of claim 1 capable of inward bending, outward bending, projection and retraction in a range about 0.4 mm by the action of force or vacuum to dynamically adjust to the variable shape of said lens tissue fragments present at said aspiration opening.

4. The elastic deformable portion of claim 1 further including gas pockets to improve the dynamic adaptive properties to provide better dynamic adjustment to the shape of said lens tissue fragments at the inner edge of said aspirating opening.

5. The elastic deformable portion of claim 1 further including differential polymerization portions to improve the dynamic adaptive properties to provide better dynamic adjustment to the shape of said lens tissue fragments at the inner edge of said aspirating opening.

6. The elastic deformable portion of claim 1 further including progressive thinning towards a distal end to improve the dynamic adaptive properties to provide better dynamic adjustment to the shape of said lens tissue fragments at the inner edge of said aspirating opening.

7. The elastic deformable portion of claim 1 capable of rapid achievement and sustaining of occlusion when said lens tissue fragments are present at said aspiration opening by narrowing the gaps between said lens tissue fragments ant the inner edge of said aspirating opening.

8. The elastic deformable portion of claim 1 capable of rapid achievement and sustaining of vacuum when said lens tissue fragments are present at said aspiration opening because of the enhanced occlusion.
